(19) United States
(12) Patent Application Publication
(10) Pub. No.: US 2009/0317502 A1
(43) Pub. Date: Dec. 24, 2009

(54) DANDRUFF TREATMENT COMPOSITIONS WITH ANTI-INFLAMMATORY AGENTS INCLUDING BOTANIC SEED OILS

(75) Inventor: Charles E. Crutchfield, III, Eagan, MN (US)

Correspondence Address: BRIGGS AND MORGAN P.A., 2200 IDS CENTER, 80 SOUTH 8TH ST, MINNEAPOLIS, MN, 55402, US

(73) Assignee: Cuticeuticals, Inc., St. Paul, MN (US)

(21) Appl. No.: 12/475,968

(22) Filed: Jun. 1, 2009

Related U.S. Application Data
(63) Continuation-in-part of application No. 11/930,622, filed on Oct. 31, 2007.
Application No. 11/930,622 is a continuation-in-part of application No. 10/462,458, filed on Jun. 16, 2003, now abandoned.
Application No. 10/462,458 is a continuation-in-part of application No. 09/882,965, filed on Jun. 15, 2001, now Pat. No. 6,579,512.
(60) Provisional application No. 61/057,316, filed on May 30, 2008.

(51) Int. Cl. A61K 36/00; A61K 36/45; A61K 36/73; A61K 36/87; A61K 36/42

(52) U.S. Cl. 424/776; 424/732; 424/765; 424/766; 424/758

(57) ABSTRACT

Compositions including a topical steroid, a blend of concentrated botanic seed oils, and a suitable vehicle for topical application to the skin are described. Compositions of the present invention can be used as a therapeutic method for treating skin conditions, including scalp conditions such as dandruff. The compositions are topically administered to the affected area of the skin of a mammal in need of such therapy.

The botanic seed oils used in the compositions may be prepared using a cold press method, and may include three or more botanic seed oils. The blend of botanic seed oils may include, for example, red raspberry seed oil, black raspberry seed oil, pomegranate seed oil, blueberry seed oil, pumpkin seed oil, carrot seed oil, blackberry seed oil, grape seed oil, cranberry seed oil, and black cumin seed oil.

### CROSS REFERENCE TO RELATED APPLICATIONS

This application claims the benefit of U.S. Provisional Application No. 61/957,316 filed May 30, 2008 and is a continuation-in-part of U.S. patent application Ser. No. 11/930,622, filed Oct. 31, 2007, which is a continuation-in-part of U.S. patent application Ser. No. 10/462,458 filed Jun. 16, 2003, now abandoned, which is a continuation-in-part of U.S. patent application Ser. No. 09/882,965 filed Jun. 15, 2001, now U.S. Pat. No. 6,579,512; the contents of which applications are hereby incorporated by reference in their entireties.

### BACKGROUND OF THE INVENTION

The present invention relates to compositions for the treatment of symptoms associated with the scalp, particularly dandruff, but also including seborrheic dermatitis, eczema, itch, and psoriasis. The composition of the invention includes cold processed botanic seed oils used in conjunction with a corticosteroid and/or salicylic acid, to treat the above scalp conditions. The composition may be used, for example, as an anti-inflammatory composition, an anti-itch liquid, an anti-eczema composition, and an external analgesic.

Dandruff affects approximately 20% of all people worldwide. In the United States, it is estimated to affect about 40-60 million. Dandruff, with its flaking, itching, scaling and redness afflicting either scalp or face, is most commonly the result of seborrhea (or seborrheic dermatitis, the medically correct terminology).

Dandruff is treated with conventional over-the-counter (“OTC”) non-prescription medical products and prescription medical products. OTC products used to treat dandruff include topical liquids that often contain either a mild hydrocortisone or salicylic acid, and can be formulated as a shampoo. The Scalpicin® brand of scalp treatment liquids, described below, are examples of OTC products that are not shampoos. Scalpicin® Anti-Itch Liquid Hypoallergenic Formula is an OTC product that uses 3.0% salicylic acid as the active ingredient along with inactive ingredients: aloe barbadensis miller, menthol, propylene glycol, SD alcohol 40B, tea tree oil (melaleuca alternifolia), tocopheryl acetate (Vitamin E), and water to dissolve the dandruff flakes commonly referred to as scales. Maximum Strength Scalpicin® Anti-Itch Liquid Dermatology Formula is an OTC product that uses 1% hydrocortisone as the active ingredient. Both of the Scalpicin® brand of dandruff treatment products, either with 1% hydrocortisone or 3.0% salicylic acid as the active ingredient, can be applied immediately after shampooing or any time of the day when scalp itch flares up.

Other OTC products include medical shampoo formulations that reduce inflammation by reducing the population of Pityrosporum ovale (or P. ovale), a fungus, that is known to exacerbate dandruff. An example of such an OTC shampoo is a non-prescription strength ketoconazole shampoo formulation used to control flaking, scaling, and itching of the scalp caused by dandruff. Ketoconazole is in a class of antifungal medications called imidazoles, and works by slowing the growth of fungi. An OTC brand of ketoconazole shampoo for dandruff sold under Nizoral® A-D includes 1% ketoconazole, as the active ingredient, with inactive ingredients of water, sodium laureth sulfate, cocamide MEA, sodium cocoyl sarcosinate, glycol distearate, acryl acid polymer (carbomer 1342), fragrance, sodium chloride, tetrasodium EDTA, butylated hydroxytoluene, quaternium-15, polyquaternium-7, sodium hydroxide and/or hydrochloric acid, and FD&C Blue No. 1.

Examples of other OTC medical shampoos for dandruff include Selsun Blue® and Head and Shoulders® that contain zinc pyrithione as an active ingredient. Still other OTC shampoos contain salicylic acid as the active ingredient that loosens up the dandruff flakes to rinses the flakes out with the shampoo. Other OTC products to treat dandruff include tar-based shampoos such as Neutrogena® T/Gel® and Selsun Gold™.

With respect to prescription medical products to control dandruff, the formulations can use the same topical liquids, but add a prescription strength steroid or prescription strength anti-fungal agent. The Derma-smooth/FS® brand of prescription scalp oil uses a mild steroid, fluocinolone acetonide topical oil 0.01%, as the active ingredient along with peanut oil as the vehicle. The Derma-smooth/FS® brand of prescription scalp oil is an effective prescription dandruff treatment. The Derma-smooth/FS® brand of scalp oil contains fluocinolone acetonide (6a, 11β, 16a)-6,9 difluoro-11,21-dihydroxy-16,17[(1-methylethylidene)bis(oxy)]-pregna-1,4-diene-3,20-dione, cyclic 16,17 acetal with acetone, a synthetic corticosteroid for topical dermatologic use. The Derma-smooth/FS® brand of scalp oil is applied as a thin layer, massaged well into the scalp, and the scalp is covered with a shower cap. The Derma-smooth/FS® brand of scalp oil requires a minimum of 4 hours on the scalp or overnight before washing off. Then the patient needs to shampoo with a non-medicated shampoo and rinse thoroughly. The process is repeat daily until desired results are obtained.

There are a couple of problems with the Derma-smooth/FS® brand of prescription scalp oil. One is that the dandruff patient needs to sleep with a shower cap covering the applied scalp oil, or at least wear the shower cap for four hours before shampooing and rinsing the scalp oil off. This is inconvenient and patients report the application procedure is annoying and uncomfortable. Another problem is the use of peanut oil as a vehicle with the steroid. While effective as a vehicle, peanut oil carries negative connotations for people with peanut allergies. Research has shown that the proteins in the peanut oil vehicle are really low allergens; and maybe don't even react at all. However, even reassurances that the peanut oil will not react negatively, or cause any harmful reaction in a patient or a patient's child suffering from a peanut allergy, is frequently met with extreme resistance from patients with these fears, and rejected as a solution.

Other examples of prescription scalp formulations for dandruff with topical steroids as the active ingredient include products with 0.05% fluocinonide, a class 2 steroid, as the active ingredient sold under the Lidex® brand, or 0.05% betamethasone dipropionatea, a class 1 steroid, sold under the Diprolene® brand. A non-steroid prescription product is the Nizorol® brand of dandruff shampoo that uses 2% ketoconazole, a prescription strength of the antifungal agent as the active ingredient.

The compositions currently used to treat dandruff and other scalp conditions, while useful, are not without problems. There is a need for improved compositions for treating multiple causes or symptoms of dandruff and scalp conditions. The available compositions often treat only one component of the many symptoms or causes of dandruff. For example, a composition can contain a topical steroid to treat the inflammation that contributes to the dandruff. A composition can contain ketoconazole that kills the fungus that contributes to the condition. A composition can contain salicylic acid that exfoliates the hyperkeratosis (thick scale), another part of the problem with dandruff. A scalp treatment composition can also contain an oil to hydrate the scalp because dry scalp can be part of the problem.

Another problem with the above treatment options for dandruff is that there is no system of OTC products that can be used conveniently to control and maintain the treatment of dandruff. Yet another problem is the need for improved dandruff compositions without peanut oil to achieve greater patient acceptance. Another problem is that the lack of intense OTC overnight oil treatment product with greater patient acceptance and effectiveness than the currently available products.

### SUMMARY OF THE INVENTION

The present invention is directed to compositions including an effective amount of a topical steroid for the treatment of skin conditions, including scalp conditions such as dandruff, in over-the-counter strength or prescription strength; a blend of concentrated botanic seed oils; and a suitable vehicle for topical application to the skin. The botanic seed oils may be prepared using a cold press method. The blend of botanic seed oils may include three or more botanic seed oils.

In some embodiments of the present invention, the topical steroid is a corticosteroid and is present in the composition in an amount ranging from about 0.01% (w/w) to about 2.5% (w/w). In some embodiments, the blend of botanic seed oils is present in the composition in an amount ranging from about 1% (w/w) to about 25% (w/w).

The blend of botanic seed oils may be made according to the following formulation: about 88% to about 98.5% (w/w) of red raspberry seed oil or black raspberry seed oil, or any combination of red raspberry seed oil and black raspberry seed oil that totals about 88% to about 98.5% (w/w) of the blend, mixed with equal amounts, about 0.25% to about 1.5% (w/w) each, of other botanic seed oils selected from pomegranate seed oil, blueberry seed oil, pumpkin seed oil, carrot seed oil, blackberry seed oil, grape seed oil, cranberry seed oil, and black cumin seed oil. This blend of botanic seed oils may be used to create each of the OTC and prescription compositions described in this application.

In addition, the compositions of the present invention can further include from about 1% to about 3% (w/w) salicylic acid for over-the-counter compositions, or from about 1% to about 10% (w/w) salicylic acid for prescription compositions.

The compositions of the invention, over-the-counter and prescription, can be used at a predetermined frequency, such as daily or weekly, as a therapeutic method for treating skin conditions, such as scalp conditions, of a mammal in need of such therapy. The compositions are topically administered to the affected area of the skin of a mammal in need of such therapy.

The compositions of the invention can be used as a system in a kit including over-the-counter compositions including the blend of botanic seed oils incorporated into: an OTC daily application product with an OTC steroid and vehicle suitable for topical application to the scalp; an OTC weekly application product with an OTC steroid oil based vehicle suitable for topical application to the scalp; an OTC shampoo without active ingredients other than the blend of botanic seed oils; and with or without an OTC hair conditioner without active ingredients other than the blend of botanic seed oils.

An object and advantage of the present invention is a composition for treating skin conditions that includes both a corticosteroid and a blend of botanic seed oils.

Another object and advantage of the present invention is a composition for treating skin conditions that includes both salicylic acid and a blend of botanic seed oils.

Another object and advantage of the present invention is a composition for treating dandruff that does not include peanut oil.

Another object and advantage of the present invention is a system for treating dandruff including: the use of a daily application product including a corticosteroid and a blend of botanic seed oils, the use of a weekly application product including a corticosteroid and a blend of botanic seed oils; the use of a shampoo including a blend of botanic seed oils; and, optionally, the use of a conditioner including a blend of botanic seed oils.

Other advantages will be understood from reading the Detailed Description of the Preferred Embodiments.

The foregoing has outlined rather broadly the features and technical advantages of the present invention in order that the detailed description of the invention that follows may be better understood. Additional features of the invention which form the subject of the claims of the invention will be described hereinafter. It should be appreciated by those skilled in the art that the specific embodiments disclosed may be readily utilized as a basis for modifying or designing other methods or compositions for carrying out the same purposes of the present invention. It should also be realized by those skilled in the art that such equivalent compositions do not depart from the spirit and scope of the invention as set forth in the appended claims. The novel features which are believed to be characteristic of the invention, both as to its composition and method of operation, together with further objects and advantages will be better understood from the following description.

### DETAILED DESCRIPTION OF THE PREFERRED EMBODIMENTS

All of the compositions of the present invention described in this Detailed Description use concentrated plant seed oils, also referred to as botanic seed oils, prepared according to a cold press method. Plant seed oils are an excellent source of antioxidants. In addition to traditional antioxidants, such as vitamins C and E, plant seed oils contain phenolic compounds which are excellent free radical scavengers. Black raspberry and red raspberry seed oils have a diversity and ultra-rich content of antioxidants, including 4 different forms of Vitamin E (Alpha and Gamma Tocopherol, Beta and Gamma Tocotrienol). These raspberry seed oils contain Omega 3 and Omega 6. In U.S. Patent Publication No. 2007/0243310, Synergistic super potent antioxidant cold pressed botanic oil blendsto Leonard et al., published Oct. 18, 2007, the inventors describe blends of seed oils as having a synergistic antioxidant effect. U.S. Patent Publication No. 2007/0243310 is incorporated by reference herein in its entirety, as though it were repeated herein.

Concentrated seed oils are described as immunostimulants in U.S. Patent Publication No. 2007/0128301, Immune Enhancement by Seed Oil and/Or Seed Flour, to Saltzman et al., published Jun. 7, 2007, and for use in treatment of cancer in Patent Publication No. 2005/0244375, Composition and Method of Cancer Treatment, to Leonard et al., published Nov. 3, 2005. In Saltzman et al., the applicant theorized that concentrated seed oils possess anti-inflammatory properties. The above referenced U.S. Patent Publication Nos. 2007/0128301 and 2005/0244375 are each incorporated by reference herein in its entirety, as though it were repeated herein.

The use of concentrated seed oils in the composition acts as natural emollients to prevent dryness and protect the skin, acting as a barrier and healing agent, as well as a soothing and softening agent of the skin. The concentrated seed oils in the composition can reduce roughness, cracking and irritation. The use of seed oil emollients nourish the skin with concentrated nutrients that can be beneficial in treating inflammatory conditions of the skin such as acne, psoriasis, eczema and rosacea. The antioxidant properties of botanic seed oils may prove to have some of the most effective rejuvenating properties of any known skin treatment to date. Charles E. Crutchfield III, M.D. Assoc. Prof. of Dermatology, University of Minnesota, et al., The Use of Nature Fresh Cold Pressed Seed Oils for Skin and Personal Care Products—A New Approach, [unpublished paper].

The article The Use of Nature Fresh Cold Pressed Seed Oils for Skin and Personal Care Products—A New Approach discusses research showing that synergistic botanic seed oils can inhibit Cox-2 activity. Cox-2 is an enzyme linked to inflammation and inflammation is associated with many skin disorders ranging from sunburn to roseacea, psoriasis, acne and dandruff. In addition, the article discusses research that synergistic botanic seed oils have potent antimicrobial activity. Many skin disorders are known to result from or be exacerbated by bacteria and fungi living on the skin surface.

A preferred method of preparing the seed oils, used in the invention, is described in U.S. Patent Publication No. 2007/0128301, Immune Enhancement by Seed Oil and/Or Seed Flour, Saltzman et al., published Jun. 7, 2007, which is incorporated by reference herein in its entirety, as though it were repeated herein. That description is:

“The oils for the composition are prepared from seeds which have been carefully dried and cleaned at temperatures below 120 degrees F. In a cold press process, the seeds are fed through the press and put under high pressure with no extra heat during the pressing process. Oil temperatures during extraction are typically 70 degrees to 90 degrees F. To insure minimal or no oxidation and the highest potential antioxidant level of the oils, the press head and oil extraction chamber can be enclosed within an inert atmosphere. Refining or removal of suspended solids and container filling can also be done in an inert atmosphere to preserve quality.”

U.S. Patent Publication No. 2007/0128301, paragraph [0016].

Not to be bound by theory, but blends of concentrated botanic seed oils made via the cold process method in the embodiments of the present invention are theorized to work with the active ingredients to increase the healing effectiveness of the active ingredient or ingredients in the composition when used for scalp conditions. The blend of botanic seed oils prepared according to a cold press method are theorized to have anti-inflammatory action apart from or in conjunction with the active ingredients.

The topical steroids (corticosteroids) used in the compositions of the invention are selected from over-the counter strengths or prescription strengths of corticosteroids. These classifications of corticosteroids are well known.

For example, an accepted method of classifying the strength of topical corticosteroid preparations is a seven-level system, in which the levels are often referred to as classes or groups. Hengge et al., “Adverse Effects of Topical Glucocorticosteroids,” Journal of the American Academy of Dermatology, January 2006, pages 1-15 (page 4 contains a detailed Table I with groups and potency). The seven levels are based on an assay measuring the anti-inflammatory properties of the topical corticosteroid, specifically the ability to induce vasoconstriction of the small blood vessels in the upper dermis. The products are subsequently tabulated into one of seven groups based on the assay results, with group I being the strongest and group VII being the weakest (see e.g. Table 1 below, which identifies two preparations from each class or group).

TABLE 1
Example of Topical steroid Preparations In Each Group

| Group No. | Generic Name | Brand Names |
|---|---|---|
| I | Clobetasol propionate, ointment, cream, 0.05%; | TEMOVATE, DERMOXIN |
| | Halobetasol propionate, ointment, cream, 0.05%; | ULTRAVATE |
| II | Amcinonide, ointment, 0.1% | CYCLOCORT |
| | Halcinonide, cream, 0.1% | HALOG |
| III | Triamcinolone acetonide, cream, 0.5% | ARISTOCORT HP |
| | Halcinonide, ointment, 0.1% | HALOG |
| IV | Fluocinolone acetonide, ointment, 0.025% | SYNALOR |
| | Halcinonide, ointment, 0.025% | HALOG |
| V | Hydrocortisone valerate, cream, 0.2% | WESTCORT |
| | Triamcinolone acetonide, lotion, 0.1% | Kenalog |
| VI | Desonide, cream, 0.05% | DESOWN, TRIDESILON |
| | Triamcinolone acetonide, cream 0.1% | ARISTOCORT |
| VII | Hydrocortisone, 0.5%, 1%, 2.5% | HYTONE, others |
| | Methylprednisolone, 1% | MEDROL |

The Hengge et al. article lists several corticosteroid preparations in each group, treatment guidelines, and adverse effects; this article is incorporated by reference herein in its entirety, as though repeated herein. The over-the-counter topical steroids are the steroids that are classified in Group VII of the Hengge et al. article.

The present invention is directed to compositions including an effective amount of a topical steroid for the treatment of skin conditions, a blend of concentrated botanic seed oils, and a suitable vehicle for topical application to the skin. Preferably, the suitable vehicle for topical application does not include peanut oil. In some embodiments of the present invention, the topical steroid is a corticosteroid and is present in the composition in an amount ranging from about 0.01% (w/w) to about 2.5% (w/w). The preferred percentage of corticosteroid used varies according to the strength of the corticosteroid. For example, the amount of steroid used can be increased if a relatively weak steroid is used, to compensate for weaker activity. Examples of corticosteroids that may be used include, but are not limited to, hydrocortisone and forms of triamcinolone, such as triamcinolone and triamcinolone acetonide.

In some embodiments, the blend of botanic seed oils is present in the composition in an amount ranging from about 1% (w/w) to about 25% (w/w). Preferably, the blend of botanic seed oils is present in an amount ranging from about 1% (w/w) to about 10% (w/w).

Blends of two or more of the concentrated seed oils are preferred to use of a single seed oil. A preferred composition includes a blend comprising three or more concentrated seed oils.

The blend of botanic seed oils may be made according to the following formulation: about 88% to about 98.5% (w/w) of red raspberry seed oil or black raspberry seed oil, or any combination of red raspberry seed oil and black raspberry seed oil that totals about 88% to about 98.5% (w/w) of the blend, mixed with equal amounts, about 0.25% to about 1.5% (w/w) each, of other botanic seed oils selected from pomegranate seed oil, blueberry seed oil, pumpkin seed oil, carrot seed oil, blackberry seed oil, grape seed oil, cranberry seed oil, and black cumin seed oil.

A first over the counter (“OTC”) composition for the treatment of scalp conditions is a formulation that includes an effective amount of an OTC topical steroid for treatment of dandruff as an active ingredient, and about 2% (w/w) blend of botanic seed oils prepared according to a cold press method, combined with a suitable topical vehicle for application to the scalp.

An embodiment of an OTC composition for treating scalp conditions is a formulation that includes about 0.25-1.0% hydrocortisone (w/w) as an active ingredient, about 2% (w/w) blend of botanic seed oils prepared according to a cold press method, and about 97-97.75% (w/w) of a suitable topical vehicle for application to the scalp.

There are several suitable topical vehicles that can be used for applying a topical steroid or other active ingredients, such as salicylic acid, to the scalp of a mammal in need of treatment. A few example of suitable topical vehicles are described herein with reference to trademarked products, and also with the ingredients of the trademarked products listed thereon. The specific vehicles used in the compositions of the invention can be any conventional formulation suitable for topical application for scalp conditions.

The botanic seed oils used in the blend of botanic seed oil for the compositions disclosed in this application are preferably prepared according to the cold press method. The major botanic seed oil component of the 2% (w/w) blend in the first composition includes about 98 to 98.5% (w/w) of red raspberry seed oil or black raspberry seed oil, or any combination of red raspberry seed oil and black raspberry seed oil that totals about 98 to 98.5% (w/w) of the blend. The raspberry seed oil is mixed with equal amounts, about 0.37 to 0.50% (w/w) each, of pomegranate seed oil, blueberry seed oil, pumpkin seed oil, and carrot seed oil.

### EXAMPLE 1

### Example of OTC Formulation

An embodiment of the first OTC composition was formulated using:

1. About 98 cc of Scalpicin® Anti-Itch Liquid Dermatology Formula that contains 1% hydrocortisone as an active ingredient, with inactive ingredients listed as SD alcohol 40-B, water, propylene glycol, menthol, tocopheryl acetate, aloe barbadensis leaf juice, melaleuca alternifolia (tea tree) leaf oil, and disodium EDTA, a suitable topical vehicle; and

2. About 2 cc of a blend of botanic seed oils prepared according to a cold press method with about 98.5% (w/w) of red raspberry seed oil, and about 0.37% (w/w) of pomegranate seed oil, about 0.37% (w/w) of blueberry seed oil, about 0.37% (w/w) of pumpkin seed oil, and about 0.37% (w/w) of carrot seed oil.

The Scalpicin® Anti-Itch Liquid Dermatology Formula includes the hydrocortisone and the suitable topical vehicle of the OTC scalp treatment composition of the present example. The inactive ingredient formulation of the Scalpicin® Anti-Itch Liquid Dermatology Formula is an example of a suitable topical vehicle. The Scalpicin® Anti-Itch Liquid Dermatology Formula is mixed with 2% (w/w) blend of botanic seed oils to create the OTC scalp treatment composition.

A therapeutic method for treating a scalp condition comprises administering the first OTC composition to the scalp of a mammal in need of such therapy. The first OTC composition is preferably applied daily to the scalp to treat the scalp condition.

The topical steroid, hydrocortisone, in the first OTC composition is merely one of a variety of acceptable steroids that can be used in OTC products. Hydrocortisone is a preferred OTC strength topical steroid for OTC compositions of the invention because it is well accepted, and used in many products.

A person of skill in the art can substitute a different OTC strength topical steroid and adjust the percentage of the steroid for an equivalent active ingredient, and adjust the percentage of the vehicle for application to the scalp accordingly, without deviating from the invention disclosure to produce an OTC product in accordance with this disclosure. The equivalents are well known to persons of skill in the art, and the invention contemplates and claims their use. See Hengge et al. article, discussed above. Thus, while the topical steroid hydrocortisone of OTC strength is described in the above example, other steroids, with other strengths, may be used in accordance with the present invention.

A prescription composition in accordance with the present invention may also include 2% (w/w) blend of botanic seed oils prepared according to a cold press method, namely about 98 to 98.5% (w/w) of red raspberry seed oil or black raspberry seed oil, or any combination of red raspberry seed oil and black raspberry seed oil that total about 98 to 98.5% (w/w) of the blend, mixed with equal amounts, about 0.37 to 0.50% (w/w) each, of pomegranate seed oil, blueberry seed oil, pumpkin seed oil, and carrot seed oil.

A second OTC composition for the treatment of scalp conditions is a formulation that includes an effective amount of an OTC topical steroid for treatment of dandruff as an active ingredient, about 1-3% (w/w) salicylic acid as an active ingredient, about 2% (w/w) blend of botanic seed oils prepared according to a cold press method, and a suitable topical vehicle for application to the scalp.

An embodiment of the second OTC composition for the treatment of scalp conditions is a formulation that includes about 0.25-1.0% (w/w) hydrocortisone and about 1-3% (w/w) salicylic acid as active ingredients; about 2% (w/w) blend of botanic seed oils prepared according to a cold press method, namely about 98 to 98.5% (w/w) of red raspberry seed oil or black raspberry seed oil or any combination of red raspberry seed oil and black raspberry seed oil that total about 98 to 98.5% (w/w) of the blend, mixed with equal amounts, about 0.37 to 0.50% (w/w) each, of pomegranate seed oil, blueberry seed oil, pumpkin seed oil, and carrot seed oil, and combined with a suitable oil based topical vehicle (without peanut oil) for application to the scalp.

### EXAMPLE 2

### Prophetic Example of an OTC Formulation

An embodiment of the second OTC composition can be formulated by using:

1) A concentration selected between the range of about 0.25-1.0% (w/w) hydrocortisone powder (w/w) added to Neurogena® Body Oil (Light Sesame Formula) to create a composition of about 0.25-1.0% (w/w) hydrocortisone per cc in Neutrogena® Body Oil (Light Sesame Formula). In this prophetic example:

1% hydrocortisone oil is the selected concentration to be made by mixing about 1 gram of hydrocortisone powder with 100 cc of Neutrogena® Body Oil (Light Sesame Formula) to create 1% (w/w) hydrocortisone per cc of this mixture;

2) A concentration selected between the range of about 1-3% (w/w) salicylic acid powder (w/w) to Neurogena® Body Oil to create a 0.25-1.0% (w/w) cc of salicylic acid in a Neutrogena® Body Oil (Light Sesame Formula) vehicle, and in this prophetic example:

1% salicylic acid is the selected concentration to be made by mixing about 1 gram of salicylic acid powder with 100 cc of Neutrogena® Body Oil (Light Sesame Formula) to create a 1% (w/w) salicylic acid per cc of this mixture;

3) Using the above prophetic example taking 1 cc of the 1% hydrocortisone and Neutrogena® Body Oil (Light Sesame Formula) mixture from step 1, and taking 1 cc of the 1% (w/w) salicylic acid and Neutrogena® Body Oil (Light Sesame Formula) mixture from step 2, and mixing both of these active ingredient mixtures with 96 cc of Neutrogena Body Oil (Light Sesame Formula) to arrive at a mixture of 98 cc; and

4) Mixing in about 2 cc of the botanic seed oil blend previously described above in Example 1, with the mixture created in step 3 to create an embodiment of the second composition.

Neutrogena® Body Oil (Light Sesame Formula), used in the prophetic example, is a suitable OTC vehicle without peanut oil for application to the scalp. Neutrogena®0 Body Oil (Light Sesame Formula) lists the following ingredients: isopropyl myristate, sesame seed oil (sesamum indicum), PEG-40 sorbitan peroleate, propylparaben, and BHT. This vehicle has no peanut oil in the formulation, which probably increases its acceptability from the perspective of patients with concerns about products using peanut oil.

A therapeutic method for treating a scalp condition comprises administering the second OTC composition to the scalp of a mammal in need of such therapy. The second OTC composition is preferably applied to the scalp on a weekly basis to treat the scalp condition.

As mentioned in the Background section, most compositions only treat a single symptom or cause of scalp conditions. But in a preferred embodiment, discussed above, the second OTC composition includes a steroid, botanical oil, and salicylic acid for treating multiple symptoms or causes of dandruff, seborrheic dermatitis, and other inflammatory conditions of the scalp.

An embodiment of a third OTC composition for the treatment of scalp conditions is a formulation that includes about 1.0% (w/w) hydrocortisone; about 9% (w/w) blend of botanic seed oils; and about 90% (w/w) of a suitable topical vehicle.

### EXAMPLE 3

### Example of an OTC Formulation

An embodiment of the third OTC composition can be formulated by mixing:

1) 1.00 grams hydrocortisone micronized powder;

2) 90.00 grams Neutrogena® Body Oil (Light Sesame Formula) (fragrance free); and

3) 9.00 grams botanic seed oil blend (formulation provided in Table 2).

The resulting composition includes about 1% (w/w) hydrocortisone, about 9% (w/w) botanical oil blend, and about 90% suitable topical vehicle, which is the Neutrogena® Body Oil (Light Sesame Formula). Neutrogena® Body Oil (Light Sesame Formula) lists the following ingredients: isopropyl myristate, sesame seed oil (sesamum indicum), PEG-40 sorbitan peroleate, propylparaben, and BHT.

The formula used to make 100 grams of the botanic seed oil blend used in this example is set forth in Table 2.

TABLE 2
Botanical Seed Oil Blend (per 100 grams)

| Botanic Seed Oil | Amount (in grams) |
|---|---|
| Red Raspberry Seed Oil | 91 |
| Blueberry Seed Oil | 1 |
| Blackberry Seed Oil | 1 |
| Carrot Seed Oil | 1 |
| Chardonnay Grape Seed Oil | 1 |
| Black Raspberry Seed Oil | 1 |
| Cranberry Seed Oil | 1 |
| Pomegranate Seed Oil | 1 |
| Pumpkin Seed Oil | 1 |
| Black Cumin Seed Oil | 1 |

A therapeutic method for treating a scalp condition comprises administering the third OTC composition to the scalp of a mammal in need of such therapy. The third OTC composition may be left on a user overnight, under a shower cap, and washed out in the morning.

A fourth OTC composition for treatment of a scalp condition includes about 2% (w/w) blend of botanic seed oils prepared according to a cold press method, combined with a suitable shampoo having no active ingredients. The blend of botanic seed oils is the same blend used in Example 1 above, namely about 98 to 98.5% (w/w) of red raspberry seed oil or black raspberry seed oil or any combination of red raspberry seed oil and black raspberry seed oil that total about 98 to 98.5% (w/w) of the blend, mixed with equal amounts, about 0.37 to 0.50% (w/w) each, of pomegranate seed oil, blueberry seed oil, pumpkin seed oil, and carrot seed oil.

### EXAMPLE 4

### Example of OTC Composition—OTC Shampoo

An embodiment of the fourth OTC composition was formulated as follows:

1) Mixing about a 2% (w/w) blend of botanic seed oils prepared according to a cold press method, the blend of botanic seed oils namely about 98.5% (w/w) of red raspberry seed oil mixed with equal amounts, about 0.37% (w/w) each, of pomegranate seed oil, blueberry seed oil, pumpkin seed oil, and carrot seed oil; with 2) 98% (w/w) Free and Clear® Shampoo (sensitive skin and scalp).

Free and Clear® Shampoo (sensitive skin and scalp) is a suitable shampoo having no active ingredients with the following ingredients on the label:

“purified water, disodium cocoamphodiacetate, disodium laureth sulfosuccinate, ammonium laureth sulfate, cocamidopropylamine oxide, citric acid, PEG-120 methyl glucose dioleate, PEG-12 dimethicone, tetrasodium EDTA, potassium sorbate.” [“CONTAINS NO: dyes, fragrance, masking fragrance, protein, lanolin, parabens, or formaldehyde.”]

A therapeutic method for treating scalp conditions comprises administering the third OTC composition to the scalp of a mammal in need of such therapy. The third OTC composition is preferably used daily in shampooing the scalp, and treating the scalp conditions.

A fifth OTC composition for treatment of scalp condition consists essentially of a 2% (w/w) blend of botanic seed oils prepared according to a cold press method, combined with a suitable hair conditioner having no active ingredients. The blend of botanic seed oils is the same blend used in Example 1 above, namely about 98 to 98.5% (w/w) of red raspberry seed oil or black raspberry seed oil or any combination of red raspberry seed oil and black raspberry seed oil that total about 98 to 98.5% (w/w) of the blend, mixed with equal amounts, about 0.37 to 0.50% (w/w) each, of pomegranate seed oil, blueberry seed oil, pumpkin seed oil, and carrot seed oil.

### EXAMPLE 5

### Example of OTC Composition—OTC Hair Conditioner

An embodiment of the fifth OTC composition was formulated as follows:

1) Mixing about a 2% (w/w) blend of botanic seed oils prepared according to a cold press method, the blend of botanic seed oils having about 98.5% (w/w) of red raspberry seed oil mixed with equal amounts, about 0.37% (w/w) each, of pomegranate seed oil, blueberry seed oil, pumpkin seed oil, and carrot seed oil; with

2) 98% (w/w) Free and Clear® Hair Conditioner (sensitive skin and scalp).

Free and Clear® Hair Conditioner (sensitive skin and scalp) is a suitable conditioner having no active ingredients with the following ingredients on the label: “purified Water; dicetyldimonium chloride; cetearyl alcohol; stearamidopropyl dimethylamine; propylene glycol; ceteareth-20; tocopheryl ccetate (Vitamin E); panthenol; potassium sorbate; and citric acid.”

A therapeutic method for treating scalp conditions comprises administering the fifth OTC composition to the scalp of a mammal in need of such therapy. The fifth OTC composition is preferably used daily after shampooing and rinsing the scalp, and aids in treating the scalp conditions.

An OTC treatment system for scalp conditions includes a kit having: a first OTC composition consists essentially of an effective amount of an OTC topical steroid as an active ingredient, about 2% (w/w) blend of botanic seed oils prepared according to a cold press method, namely about 98 to 98.5% (w/w) of red raspberry seed oil or black raspberry seed oil or any combination of red raspberry seed oil and black raspberry seed oil that total about 98 to 98.5% (w/w) of the blend, mixed with equal amounts, about 0.37 to 0.50% (w/w) each, of pomegranate seed oil, blueberry seed oil, pumpkin seed oil, and carrot seed oil, and combined with a suitable topical vehicle with directions to apply daily to the scalp; a second OTC composition that consists essentially of an effective amount of an OTC topical steroid for treatment of dandruff as an active ingredient; about 1-3% (w/w) salicylic acid as an active ingredient; about 2% (w/w) blend of botanic seed oils prepared according to a cold press method, namely about 98 to 98.5% (w/w) of red raspberry seed oil or black raspberry seed oil or any combination of red raspberry seed oil and black raspberry seed oil that total about 98 to 98.5% (w/w) of the blend, mixed with equal amounts, about 0.37 to 0.50% (w/w) each, of pomegranate seed oil, blueberry seed oil, pumpkin seed oil, and carrot seed oil, combined with a suitable oil based topical vehicle and directions to apply weekly to the scalp; and an OTC shampoo composition that includes a 2% (w/w) blend of botanic seed oils prepared according to a cold press method, namely about 98 to 98.5% (w/w) of red raspberry seed oil or black raspberry seed oil, or any combination of red raspberry seed oil and black raspberry seed oil that total about 98 to 98.5% (w/w) of the blend, mixed with equal amounts, about 0.37 to 0.50% (w/w) each, of pomegranate seed oil, blueberry seed oil, pumpkin seed oil, and carrot seed oil, combined with a suitable shampoo having no active ingredients and directions to apply daily to the scalp.

Another OTC treatment system for scalp conditions includes a kit having: a first OTC composition consists essentially of an effective amount of an OTC topical steroid as an active ingredient, about 2% (w/w) blend of botanic seed oils prepared according to a cold press method, namely about 98 to 98.5% (w/w) of red raspberry seed oil or black raspberry seed oil or any combination of red raspberry seed oil and black raspberry seed oil that total about 98 to 98.5% (w/w) of the blend, mixed with equal amounts, about 0.37 to 0.50% (w/w) each, of pomegranate seed oil, blueberry seed oil, pumpkin seed oil, and carrot seed oil, and combined with a suitable topical vehicle with directions to apply daily to the scalp; a second OTC composition that consists essentially of an effective amount of an OTC topical steroid for treatment of dandruff as an active ingredient; about 1-3% (w/w) salicylic acid as an active ingredient; about 2% (w/w) blend of botanic seed oils prepared according to a cold press method, namely about 98 to 98.5% (w/w) of red raspberry seed oil or black raspberry seed oil or any combination of red raspberry seed oil and black raspberry seed oil that total about 98 to 98.5% (w/w) of the blend, mixed with equal amounts, about 0.37 to 0.50% (w/w) each, of pomegranate seed oil, blueberry seed oil, pumpkin seed oil, and carrot seed oil, combined with a suitable oil based topical vehicle and directions to apply weekly to the scalp; an OTC shampoo composition that includes a 2% (w/w) blend of botanic seed oils prepared according to a cold press method, namely about 98 to 98.5% (w/w) of red raspberry seed oil or black raspberry seed oil, or any combination of red raspberry seed oil and black raspberry seed oil that total about 98 to 98.5% (w/w) of the blend, mixed with equal amounts, about 0.37 to 0.50% (w/w) each, of pomegranate seed oil, blueberry seed oil, pumpkin seed oil, and carrot seed oil, combined with a suitable shampoo having no active ingredients and directions to apply daily to the scalp; and an OTC conditioner composition that consists essentially of a 2% (w/w) blend of botanic seed oils prepared according to a cold press method, namely about 98 to 98.5% (w/w) of red raspberry seed oil or black raspberry seed oil, or any combination of red raspberry seed oil and black raspberry seed oil that total about 98 to 98.5% (w/w) of the blend, mixed with equal amounts, about 0.37 to 0.50% (w/w) each, of pomegranate seed oil, blueberry seed oil, pumpkin seed oil, and carrot seed oil, combined with a suitable conditioner with no active ingredients and directions to apply daily to the scalp after shampooing and rinsing the OTC shampoo composition from the scalp.

### EXAMPLE 6

### Example of a Prescription Strength Formulation

A sixth composition for the treatment of scalp conditions is a formulation that consists essentially of effective amount of a prescription topical steroid for treatment of dandruff as active ingredient; a 2% (w/w) blend of botanic seed oils prepared according to a cold press method, namely about 98 to 98.5% (w/w) of red raspberry seed oil or black raspberry seed oil, or any combination of red raspberry seed oil and black raspberry seed oil that total about 98 to 98.5% (w/w) of the blend, mixed with equal amounts, about 0.37 to 0.50% (w/w) each, of pomegranate seed oil, blueberry seed oil, pumpkin seed oil, and carrot seed oil; and a suitable topical vehicle for application to the scalp.

An embodiment of the sixth composition for the treatment of scalp conditions is a prescription formulation that includes about 0.025-0.1% (w/w) triamcinolone as active ingredient; a 2% (w/w) blend of botanic seed oils prepared according to a cold press method, namely about 98 to 98.5% (w/w) of red raspberry seed oil or black raspberry seed oil, or any combination of red raspberry seed oil and black raspberry seed oil that total about 98 to 98.5% (w/w) of the blend, mixed with equal amounts, about 0.37 to 0.50% (w/w) each, of a pomegranate seed oil, blueberry seed oil, pumpkin seed oil, and carrot seed oil; and about 97.975 to about 97.9% (w/w) of a suitable topical vehicle for application to the scalp.

A concentration of about 0.025-0.1% triamcinolone (w/w) topical steroid is a preferred topical steroid in the sixth composition. While about 0.025-0.1% triamcinolone (w/w/) is the prescription strength steroid used in the sixth composition, an equally effective prescription strength steroid for the scalp condition can be substituted in its place with the steroid percentage adjusted to appropriate concentrations, and the percentage of topical vehicle adjusted accordingly to achieve similar results. See Hengge et al. article, discussed above.

Another embodiment of the sixth composition for the treatment of scalp conditions is a prescription formulation that consists essentially of about 0.025-0.1% (w/w) triamcinolone and about 1% to 10% (w/w) salicylic acid as active ingredients; a 2% (w/w) blend of botanic seed oils prepared according to a cold press method, namely about 98 to 98.5% (w/w) of red raspberry seed oil or black raspberry seed oil or any combination of red raspberry seed oil and black raspberry seed oil that total about 98 to 98.5% (w/w) of the blend, mixed with equal amounts, about 0.37 to 0.50% (w/w) each, of pomegranate seed oil, blueberry seed oil, pumpkin seed oil, and carrot seed oil; combined with about 87.9 to 96.975% (w/w) of a suitable topical vehicle for application to the scalp.

As mentioned in the Background section, most compositions only treat a single symptom or cause of scalp conditions. A preferred embodiment of the sixth composition, described above, includes a steroid, blend of botanical oils, and salicylic acid for treating multiple symptoms or causes of dandruff, seborrheic dermatitis, and other inflammatory conditions of the scalp.

A therapeutic method for treating scalp conditions comprises administering the sixth composition to the scalp of a mammal in need of such therapy. The sixth composition is preferably applied to the scalp on a weekly basis to treat the scalp condition.

An embodiment of a seventh composition for the treatment of scalp conditions is a formulation that includes about 0.05% (w/w) triamcinolone; about 9% (w/w) blend of botanic seed oils; and about 90.95% (w/w) of a suitable topical vehicle.

### EXAMPLE 7

### Example of a Prescription Strength Formulation

An embodiment of the third OTC composition can be formulated by mixing:

1) 0.05 grams triamcinolone micronized powder;

2) 90.95 grams Neutrogena® Body Oil (Light Sesame Formula) (fragrance free); and

3) 9.00 grams botanic seed oil blend (formulation provided in Table 2).

The resulting composition includes about 0.05% (w/w) triamcinolone, about 9% (w/w) botanical oil blend, and about 90.95% (w/w) suitable topical vehicle, which is the Neutrogena® Body Oil (Light Sesame Formula). Neutrogena® Body Oil (Light Sesame Formula) lists the following ingredients: isopropyl myristate, sesame seed oil (sesamum indicum), PEG-40 sorbitan peroleate, propylparaben, and BHT.

The formula used to make 100 grams of the botanic seed oil blend used in this example is set forth in Table 2, in Example 3, above.

A therapeutic method for treating a scalp condition comprises administering the seventh composition to the scalp of a mammal in need of such therapy. The seventh OTC composition may be left on a user overnight, under a shower cap, and washed out in the morning.

### STUDY USING OTC COMPOSITION AND PRESCRIPTION COMPOSITIONS IN 500 PATIENT WITH DANDRUFF

Under the inventor's supervision, 500 patients with dandruff were treated with preferred embodiments of several of the compositions. The results were dramatic showing that an overall average of about 89% of the patient pool of 500 patients, treated with compositions having the blend of botanic oils, reported that the treatment improved the dandruff.

The 500 patients were broken down into three (3) groups:

1) Group 1 was composed of 225 patients treated with an embodiment of the prescription composition described in Example 6 as follows:
About 0.05% (w/w) triamcinolone formulated using the Aristicort® brand of prescription product containing the prescription steroid strength in a suitable vehicle, and mixing a 2% (w/w) blend of botanic seed oils prepared according to a cold press method, namely about 98.5% (w/w) of red raspberry seed oil, mixed with equal amounts, about 0.37% (w/w) each, of pomegranate seed oil, blueberry seed oil, pumpkin seed oil, and carrot seed oil.
   The 225 patients receiving this treatment with an embodiment of the Example 6 prescription compound were instructed to apply the above formulation weekly to the scalp.

2) Group 2 was composed of 225 patients treated with an embodiment of the prescription composition described in Example 6 as follows:
About 0.025% (w/w) triamcinolone using the Aristicort® brand of prescription product containing the prescription steroid strength in a suitable vehicle, and mixing a 2% (w/w) blend of botanic seed oils prepared according to a cold press method, namely about 98.5% (w/w) of red raspberry seed oil, mixed with equal amounts, about 0.37% (w/w) each, of pomegranate seed oil, blueberry seed oil, pumpkin seed oil, and carrot seed oil.
   The 225 patients receiving this treatment with an embodiment of the Example 6 prescription compound were instructed to apply the above formulation weekly to the scalp.

3) Group 3 was composed of 50 patients treated with an embodiment of the first OTC composition formulated as follows:
a. About 98 cc of Scalpicin® Anti-Itch Liquid Dermatology Formula that contains 1% hydrocortisone, as an active ingredient, with inactive ingredients listed as SD alcohol 40-B, water, propylene glycol, and disodium EDTA, as a suitable topical vehicle;
b. About 2 cc of blend of botanic seed oils prepared according to a cold press method with about 98.5% (w/w) of red raspberry seed oil, and about 0.37% (w/w) of pomegranate seed oil, about 0.37% (w/w) of blueberry seed oil, about 0.37% (w/w) of pumpkin seed oil, and about 0.37% (w/w) of carrot seed oil.
   The 50 patients receiving this treatment with an embodiment of the first OTC compound were instructed to apply the above formulation daily to the scalp.

The results of the groups 1-3 were that all groups reported at least a 70% average for improvement of the dandruff. The breakdown of results was that:

1) Group 1, receiving the 0.05% (w/w) triamcinolone formulated using Aristicort® brand, highest prescription strength in study, and the 2% blend of botanic seed oils, in the suitable vehicle provided in the Aristicort® brand, reported the highest improvement percentage of all three groups;

2) Group 2, receiving the 0.025% (w/w) triamcinolone formulated using Aristicort® brand, next highest prescription strength, and the 2% blend of botanic seed oils, in the suitable vehicle provided in the Aristicort® brand, reported the second highest improvement percentage of all three groups; and

3) Group 3, receiving the OTC composition described above, reported the third highest improvement percentage of all three groups.

The present invention may be embodied in other specific forms without departing from the spirit or essential attributes thereof, and it is therefore desired that the present embodiments be considered in all respects as illustrative and not restrictive, reference being made to the appended claims rather than to the foregoing description to indicate the scope of the invention.

1. A topical composition for treating a skin condition, comprising a corticosteroid and a botanic seed oil blend, wherein the botanic seed oil blend comprises three or more botanic seed oils prepared according to a cold press method.

2. The topical composition of claim 1, wherein the corticosteroid is present in an amount ranging from about 0.01% (w/w) to about 2.5% (w/w).

3. The topical composition of claim 1, wherein the corticosteroid is selected from the group consisting of clobetasol propionate, halobetasol propionate, amcinonide, halcinonide, triamcinolone, fluocinolone acetonide, hydrocortisone valerate, desonide, hydrocortisone, and methylprednisolone.

4. The topical composition of claim 1, wherein the botanic seed oil blend is present in an amount ranging from about 1% (w/w) to about 25% (w/w).

5. The topical composition of claim 1, wherein the three or more botanic seed oils are selected from the group consisting of red raspberry seed oil, blueberry seed oil, blackberry seed oil, carrot seed oil, grape seed oil, black raspberry seed oil, cranberry seed oil, pomegranate seed oil, pumpkin seed oil, and black cumin seed oil.

6. The topical composition of claim 5, wherein the botanic seed oil blend comprises red raspberry seed oil, black raspberry seed oil, blueberry seed oil, carrot seed oil, pomegranate seed oil, and pumpkin seed oil.

7. The topical composition of claim 6, wherein the combined weight percent of the red raspberry seed oil and the black raspberry seed oil ranges from about 88% (w/w) to about 98.5% (w/w) by weight of the botanic seed oil blend.

8. The topical composition of claim 5, wherein the botanic seed oil blend comprises red raspberry seed oil, blueberry seed oil, blackberry seed oil, carrot seed oil, grape seed oil, black raspberry seed oil, cranberry seed oil, pomegranate seed oil, pumpkin seed oil, and black cumin seed oil.

9. The topical composition of claim 1, further comprising salicylic acid.

10. A topical composition for treating a skin condition, comprising a corticosteroid in an amount ranging from about 0.01% (w/w) to about 2.5% (w/w), and a botanic seed oil blend in an amount ranging from about 1% (w/w) to about 25% (w/w), wherein the botanic seed oil blend comprises three or more botanic seed oils.

11. The topical composition of claim 10, wherein the corticosteroid is present in an amount ranging from about 0.01% (w/w) to about 2.5% (w/w).

12. The topical composition of claim 10, wherein the corticosteroid is selected from the group consisting of clobetasol propionate, halobetasol propionate, amcinonide, halcinonide, triamcinolone, fluocinolone acetonide, hydrocortisone valerate, desonide, hydrocortisone, and methylprednisolone.

13. The topical composition of claim 10, wherein the botanic seed oil blend is present in an amount ranging from about 1% (w/w) to about 10% (w/w).

14. The topical composition of claim 10, wherein the three or more botanic seed oils are selected from the group consisting of red raspberry seed oil, blueberry seed oil, blackberry seed oil, carrot seed oil, grape seed oil, black raspberry seed oil, cranberry seed oil, pomegranate seed oil, pumpkin seed oil, and black cumin seed oil.

15. The topical composition of claim 14, wherein the botanic seed oil blend comprises red raspberry seed oil, black raspberry seed oil, blueberry seed oil, carrot seed oil, pomegranate seed oil, and pumpkin seed oil.

16. The topical composition of claim 15, wherein the combined weight percent of the red raspberry seed oil and the black raspberry seed oil ranges from about 88% (w/w) to about 98.5% (w/w) by weight of the botanic seed oil blend.

17. The topical composition of claim 14, wherein the botanic seed oil blend comprises red raspberry seed oil, blueberry seed oil, blackberry seed oil, carrot seed oil, grape seed oil, black raspberry seed oil, cranberry seed oil, pomegranate seed oil, pumpkin seed oil, and black cumin seed oil.

18. The topical composition of claim 17, wherein the red raspberry seed oil is present in an amount ranging from about 86.5% (w/w) to about 97.75% (w/w) by weight of the botanic seed oil blend.

19. The topical composition of claim 1, further comprising salicylic acid.

20. A method for treating a skin condition comprising applying, to an affected area of the skin of a user, an effective amount of a topical composition comprising a corticosteroid and a botanic seed oil blend, wherein the botanic seed oil blend comprises three or more botanic seed oils prepared according to a cold press method.

21. The method of claim 20, wherein the corticosteroid is present in an amount ranging from about 0.01% (w/w) to about 2.5% (w/w), and wherein the botanic seed oil blend is present in an amount ranging from about 1% (w/w) to about 25% (w/w).

22. The method of claim 21, wherein the botanic seed oil blend is present in an amount ranging from about 1% (w/w) to about 10% (w/w).

23. The method of claim 20, wherein the three or more botanic seed oils are selected from the group consisting of red raspberry seed oil, blueberry seed oil, blackberry seed oil, carrot seed oil, grape seed oil, black raspberry seed oil, cranberry seed oil, pomegranate seed oil, pumpkin seed oil, and black cumin seed oil.

24. The method of claim 23, wherein the botanic seed oil blend comprises red raspberry seed oil, black raspberry seed oil, blueberry seed oil, carrot seed oil, pomegranate seed oil, and pumpkin seed oil.

25. The method of claim 24, wherein the combined weight percent of the red raspberry seed oil and the black raspberry seed oil ranges from about 88% (w/w) to about 98.5% (w/w) by weight of the botanic seed oil blend.

26. The method of claim 20, wherein the skin condition is dandruff, and the affected area is the user's scalp.
